(19) United States
(12) Patent Application Publication
(10) Pub. No.: US 2017/0332907 A1
(43) Pub. Date: Nov. 23, 2017

(54) DEVICE FOR JUDGING STATE OF MOTION OF A USER

(71) Applicant: Jarvish Inc., Taipei City (TW)

(72) Inventor: Younger Liang, Taipei City (TW)

(21) Appl. No.: 15/250,154

(22) Filed: Aug. 29, 2016

(30) Foreign Application Priority Data
May 18, 2016 (TW) 105121291

(51) Int. Cl. A61B 5/00; A61B 5/11

(52) U.S. Cl. CPC A61B 5/0024; A61B 5/681; A61B 5/7282; A61B 5/6898; A61B 5/0022; A61B 5/6803; A61B 5/1123; A61B 2562/0219

(57) ABSTRACT

A device for judging state of motion of a user is disposed on multiple carried articles carried by the user. The device includes multiple sensation modules disposed on the multiple carried articles. Each sensation module serves to generate motional state information. The device further includes a first processor. The first processor serves to receive and compare the respective motional state information. In case it is found that both the motional state information have instantaneously changed data, the first processor generates notice information. Accordingly, the device can more precisely judge the state of motion of the user to reduce waste of social resource and increase survival rate in case of an accident.

This application claims the priority benefit of Taiwan patent application number 105121291 filed on May 18, 2016.

### BACKGROUND OF THE INVENTION

### 1. Field of the Invention

The present invention relate generally to a field of judging state of motion of a user, and more particularly to a device for judging state of motion of a user.

### 2. Description of the Related Art

In the modern society, a common person often carries various carried articles such as, but not limited to, a mobile phone, a tablet, a portable media player, a personal digital assistant, a helmet, a necklace, a bracelet, a watch, a ring, clothes, a pair of glasses and shoes. Along with the high development of science and technology, a monitoring system is applied to the above carried articles to monitor the state of motion of a user. For example, by means of the above carried articles, it is monitored whether the user is collided, falls down or loses his/her conscience. In case an accident happens to the user, the sensors disposed on the carried articles will read the change of the state of motion of the user. After processed, the sensor finds that an accident happens to the user and then automatically informs a medical worker or the user's family to get to the scene and help the user. This can minimize the possibility of death of the user.

However, in the conventional application, only one carried article is equipped with the sensor to monitor the state of motion of the user. As a result, a misjudgment often takes place. For example, in the case that the carried article drops down, but the user is safe, due to the monitoring system, the sensor will still judge the situation to be fall of the user and then automatically inform a medical worker or the user's family to get to the scene. This will lead to inconvenience of the user and waste of much social resource. Therefore, the conventional technique for judging state of motion of a user has some shortcomings and needs to be improved. It has become a topic how to minimize the possibility of misjudgment so as to reduce the waste of social resource.

### SUMMARY OF THE INVENTION

It is therefore a primary object of the present invention to provide a device for judging state of motion of a user, which has higher judgment precision.

It is a further object of the present invention to provide the above device for judging state of motion of a user, which can reduce waste of social resource.

It is still a further object of the present invention to provide the above device for judging state of motion of a user, which can increase survival rate of the user in case an accident happens to the user.

To achieve the above and other objects, the device for judging state of motion of a user of the present invention is disposed on multiple carried articles carried by a user. The device for judging state of motion of a user includes: at least one first sensation module disposed on at least one of the carried articles, the first sensation module having a first sensor and a first transceiver, the first sensor serving to detect the state of motion of the at least one carried article of the multiple carried articles to generate first motional state information, the first motional state information being transmitted via the first transceiver; and a second sensation module disposed on the other of the carried articles, the second sensation module having a second sensor, a second transceiver and a first processor, the first processor being connected to the second sensor and the second transceiver, the second sensor serving to detect the state of motion of the other carried article of the multiple carried articles to generate second motional state information and transmit the second motional state information to the first processor, the first processor receiving the first motional state information of the first sensation module via the second transceiver and comparing the first and second motional state information to find that both the first and second motional state information have instantaneously changed data to generate notice information, the notice information being transmitted to a remote unit via the second transceiver. According to the above arrangement, the device for judging state of motion of a user of the present invention has higher judgment precision to reduce waste of social resource. Moreover, the device for judging state of motion of a user can increase survival rate of the user in case an accident happens to the user.

Alternatively, the device for judging state of motion of a user of the present invention is disposed on multiple carried articles carried by a user. The device for judging state of motion of a user includes: multiple sensation modules disposed on the multiple carried articles, each sensation module having a sensor and a first transceiver, the sensors serving to detect the state of motion of the multiple carried articles to generate multiple motional state information, the motional state information being transmitted via first transceivers of the sensation modules; and a processing module disposed on at least one of the multiple carried articles, the processing module having a first processor and a second transceiver connected to the first processor, the second transceiver receiving the motional state information transmitted from the first transceiver, the first processor receiving the motional state information and comparing the motional state information to find that both the motional state information have instantaneously changed data to generate notice information, the notice information being transmitted to a remote unit via the second transceiver. According to the above arrangement, the device for judging state of motion of a user of the present invention has higher judgment precision to reduce waste of social resource. Moreover, the device for judging state of motion of a user can increase survival rate of the user in case an accident happens to the user.

In the above device for judging state of motion of a user, the carried articles are a mobile device or a wearing accessory or a wear or any combination thereof.

In the above device for judging state of motion of a user, the first sensation module further has a second processor connected to the first sensor and the first transceiver. The second processor transmits the first motional state information to the second sensation module via the first transceiver.

In the above device for judging state of motion of a user, the first sensor includes a first accelerometer and a first gyroscope. The first accelerometer detects the acceleration of the at least one carried article of the multiple carried articles to generate first acceleration information. The first gyroscope detects the angular velocity of the at least one carried article of the multiple carried articles to generate first angular velocity information. The first acceleration information and the first angular velocity information are combined to generate the first motional state information.

In the above device for judging state of motion of a user, the second sensor includes a second accelerometer and a second gyroscope. The second accelerometer detects the acceleration of the other carried article of the multiple carried articles to generate second acceleration information. The second gyroscope detects the angular velocity of the other carried article of the multiple carried articles to generate second angular velocity information. The second acceleration information and the second angular velocity information are combined to generate the second motional state information.

In the above device for judging state of motion of a user, the first sensor further includes a first electronic compass, a first air pressure sensation unit, a first positioning unit or a first pickup unit. The first electronic compass serves to generate first directional information. The first air pressure sensation unit serves to generate first air pressure information. The first positioning unit serves to generate first positioning information, the first pickup unit serving to generate first audio information.

In the above device for judging state of motion of a user, the second sensor further includes a second electronic compass, a second air pressure sensation unit, a second positioning unit or a second pickup unit. The second electronic compass serves to generate second directional information. The second air pressure sensation unit serves to generate second air pressure information. The second positioning unit serves to generate second positioning information, the second pickup unit serving to generate second audio information.

In the above device for judging state of motion of a user, the remote unit is a server end or a mobile device.

In the above device for judging state of motion of a user, each sensation module has a second processor connected to the sensor and the first transceiver. The second processor transmits the motional state information to the processing module via the first transceiver.

In the above device for judging state of motion of a user, the sensor includes an accelerometer and a gyroscope. The accelerometer detects the acceleration of the carried article to generate acceleration information. The gyroscope detects the angular velocity of the carried article to generate angular velocity information. The acceleration information and the angular velocity information are combined to obtain the motional state information.

In the above device for judging state of motion of a user, the sensor further includes an electronic compass, an air pressure sensation unit, a positioning unit or a pickup unit. The electronic compass serves to generate directional information. The air pressure sensation unit serves to generate air pressure information. The positioning unit serves to generate positioning information. The pickup unit serves to generate audio information.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a block diagram of a first embodiment of the present invention;

FIG. 2 is a perspective view of the first embodiment of the present invention;

FIG. 3 is a block diagram of the first sensor of the first embodiment of the present invention;

FIG. 4 is a block diagram of the second sensor of the first embodiment of the present invention;

FIG. 5 is a schematic diagram showing the use of the first embodiment of the present invention in a first state;

FIG. 6 is a schematic diagram showing the use of the first embodiment of the present invention in a second state;

FIG. 7 is a block diagram of a second embodiment of the present invention;

FIG. 8 is a perspective view of the second embodiment of the present invention;

FIG. 9 is a block diagram of a third embodiment of the present invention;

FIG. 10 is a perspective view of the third embodiment of the present invention;

FIG. 11 is a block diagram of the sensor of the third embodiment of the present invention;

FIG. 12 is a schematic diagram showing the use of the third embodiment of the present invention in a first state; and

FIG. 13 is a schematic diagram showing the use of the third embodiment of the present invention in a second state.

### DETAILED DESCRIPTION OF THE PREFERRED EMBODIMENTS

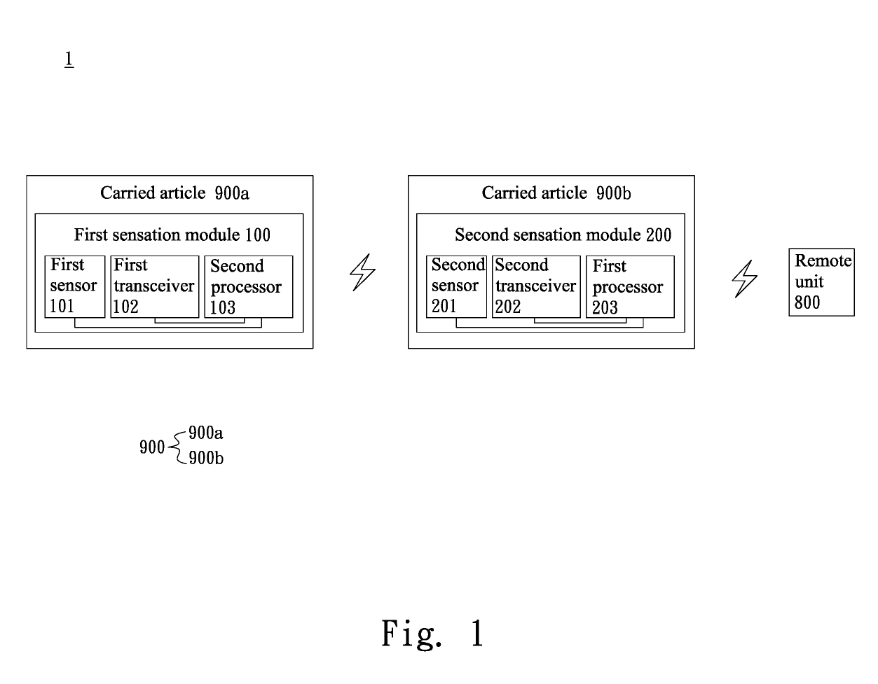

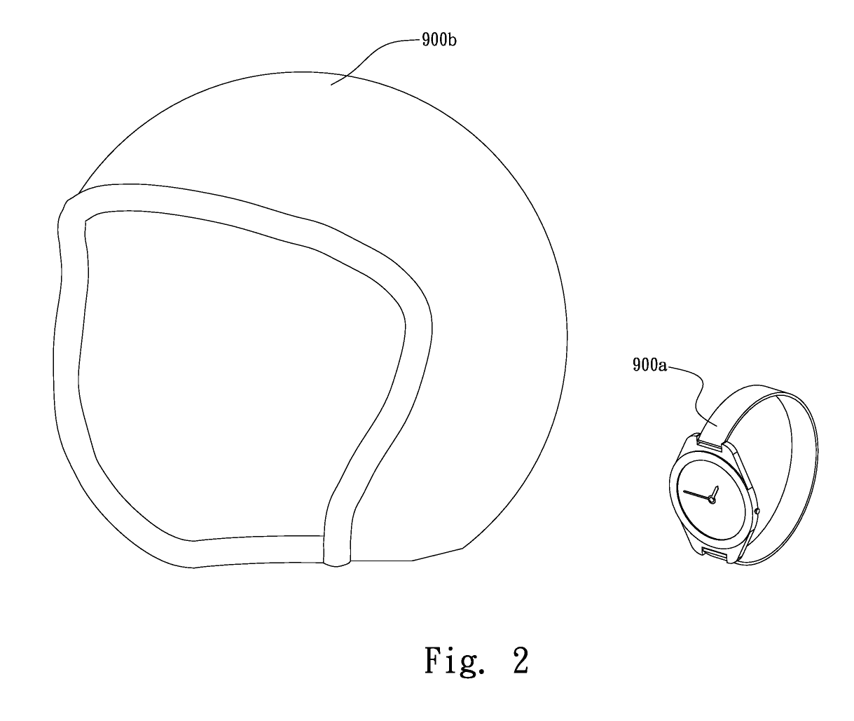

Please refer to FIGS. 1 and 2. FIG. 1 is a block diagram of a first embodiment of the present invention. FIG. 2 is a perspective view of the first embodiment of the present invention. The device 1 for judging state of motion of a user of the present invention is disposed on multiple carried articles 900 carried by a user. In this embodiment, there are two carried articles 900, one of which is a watch and the other of which is a helmet. The device 1 for judging state of motion of a user includes at least one first sensation module 100 and a second sensation module 200. The first sensation module 100 is disposed on the watch (the carried article 900a of the multiple carried articles 900), while the second sensation module 200 is disposed on the helmet (the other carried article 900b of the multiple carried articles 900). The carried articles 900 are not limited to the watch and the helmet of this embodiment. In practice, the carried articles 900 can be alternatively a mobile device (such as an intelligent mobile phone or tablet) or a wearing accessory (such as a bracelet, anklet or intelligent bracelet) or a wear (such as a site hat or necklace) or any combination of the above mobile device, wearing accessory and wear. The first and second sensation modules are respectively disposed on different carried articles. Only one of the first and second sensation modules is disposed on one carried article. That is, the first and second sensation modules will not be disposed on the same carried article.

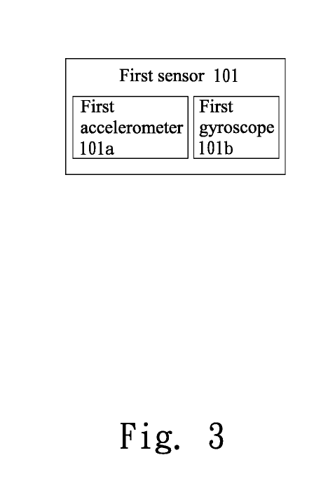

The first sensation module 100 has a first sensor 101 and a first transceiver 102. The first sensor 101 serves to detect the state of motion of the carried article 900a (the watch) of the multiple carried articles 900. For example, the first sensor 101 includes a first accelerometer 101a and a first gyroscope 101b (as shown in FIG. 3). The first accelerometer 101a detects the acceleration of the carried article 900a to generate first acceleration information. The first gyroscope 101b detects the angular velocity of the carried article 900a to generate first angular velocity information. The first sensation module 100 further includes a second processor 103 connected to the first sensor 101 and the first transceiver 102. The second processor 103 receives and combines the first acceleration information and the first angular velocity information to generate first motional state information. In practice, the second processor 103 is any operation component with operation function, such as, but not limited to, a central processing unit (CPU), a microprocessor or a digital signal processor (DSP). The first sensation module 100 transmits the first motional state information via the first transceiver 102. In this embodiment, the first transceiver 102 is, but not limited to, a Wi-Fi transceiver. In practice, the first transceiver 102 can be any transceiver component with information transmitting/receiving function, such as a Bluetooth, RFID, 3G or 4G transceiver.

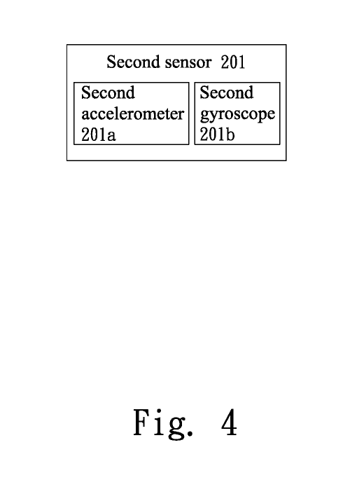

The second sensation module 200 has a second sensor 201, a second transceiver 202 and a first processor 203. The first processor 203 is connected to the second sensor 201 and the second transceiver 202. The second sensor 201 serves to detect the state of motion of the other carried article 900b (the helmet) of the multiple carried articles 900. For example, the second sensor 201 includes a second accelerometer 201a and a second gyroscope 201b (as shown in FIG. 4). The second accelerometer 201a detects the acceleration of the other carried article 900b to generate second acceleration information. The second gyroscope 201b detects the angular velocity of the other carried article 900h to generate second angular velocity information. The first processor 203 receives and combines the second acceleration information and the second angular velocity information to generate second motional state information. The first processor 203 receives the first motional state information of the first sensation module 100 via the second transceiver 202 and compares the first and second motional state information to find that both the first and second motional state information have instantaneously changed data to generate notice information. In practice, the first processor 203 is any operation component with operation function, such as a central processing unit (CPU), a microprocessor or a digital signal processor (DSP). In this embodiment, the second transceiver 202 is a Wi-Fi transceiver and the second transceiver 202 is coupled with the first transceiver 102. In practice, the second transceiver 202 can be any transceiver component with information transmitting/receiving function, such as a Bluetooth, RFID, 3G or 4G transceiver.

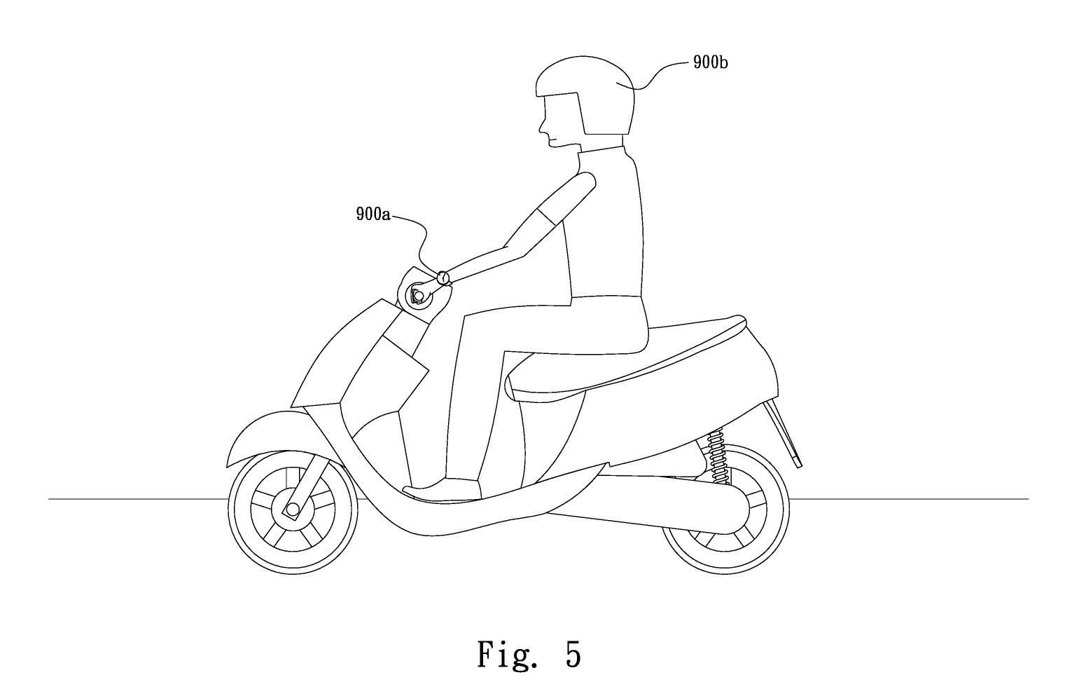

The instantaneously changed data are such that the first and second motional state information of the first and second sensors 101, 201 in normal state within a certain range. In case an accident takes place at a certain point of time, the motional states of the carried articles 900a, 900b detected by the first and second sensors 101, 201 will be compared with the normal state and found greatly changed. For example, as shown in FIG. 5, in normal state, a user wears a helmet (the other carried article 900b) and a watch (the carried article 900a) and rides a motorcycle. During the riding, the first accelerometer 101a and the first gyroscope 101b of the first sensor 101 detect that the acceleration and angular velocity of the watch periodically up and down and left and right change within a small range. Also, the second accelerometer 201a and the second gyroscope 201b of the second sensor 201 detect that the acceleration and angular velocity of the helmet periodically change within substantially in the same manner as the first sensor 101. Therefore, the first motional state information is substantially identical to the second motional state information.

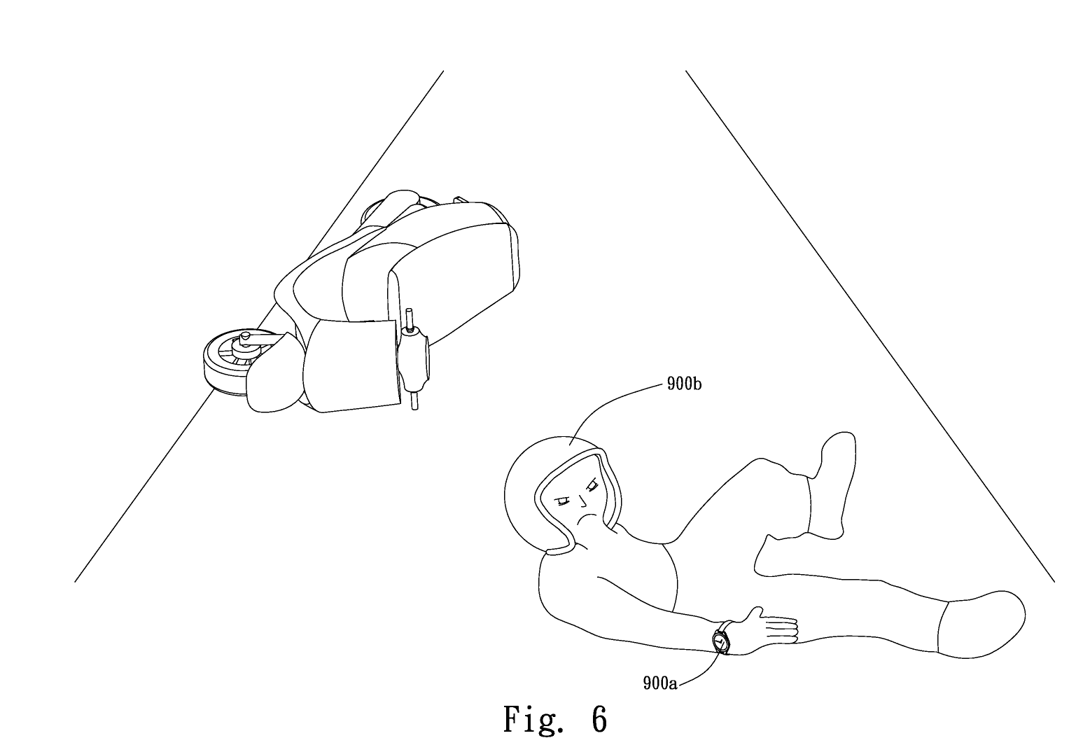

Referring to FIG. 6, in case an accident happens to the user during the riding (at a certain point of time), for example, the user is collided or falls down, the periodical change of the acceleration and angular velocity of the watch and the helmet detected by the first and second sensors 101, 201 will simultaneously greatly change. At this time, the first processor 203 of the second sensation module 200 compares and finds that at the point of the accident, the first and second motional state information simultaneously greatly change (the change amounts are not certainly identical to each other). Accordingly, it can be further judged that an accident happens to the user. Under such circumstance, the first processor 203 generates notice information and transmits the notice information to a remote unit 800 via the second transceiver 202. Preferably, the remote unit 800 is a server end and includes the mobile device or database of another user that regards the server end as an information service providing end and receives the information service from the server end. For example, in case an accident happens to the user, the device 1 for judging state of motion of a user can immediately transmit the notice information to the remote unit 800. Then, the remote unit 800 transmits the notice information to the mobile phone of the user's family or the computer of a medical station. Accordingly, the device 1 for judging state of motion of a user of the present invention can get the user a quicker medical care after the accident. This can increase the survival rate. In a modified embodiment, the remote unit 800 can be a mobile device (such as an intelligent mobile phone or a tablet). For example, the remote unit 800 can be an intelligent mobile phone of the user's family. In this case, the intelligent mobile phone of the user's family can directly receive the notice information transmitted from the second transceiver 202 on the user to let the user's family at the first moment know that an accident happens to the user and immediately help the user.

In addition, when riding a motorcycle, the user wears the helmet (the carried article 900b) on his/her head and wears the watch (the carried article 900a) on his/her hand. During the riding, one of the parts (such as the head and hand) of the user that wear the carried articles 900 may make a motion such as turn the head or raise the hand. At this time, the first processor 203 of the second sensation module 200 will compare the first and second motional state information. In case the first motional state information or the second motional state information of one of the watch and the helmet is found changed and only the sensor of one carried article 900 detects the motional state of the carried article 900 is changed, the first processor 203 will judge that no accident happens to the user since only one motional state is changed. Alternatively, in case the parts of the user that wear the carried articles 900 make a motion such as turn the head or raise the hand and the change of the first motional state information or the second motional state information due to the motion is not so much, the first processor 203 will judge that no accident happens to the user since the change of the motional state of the carried articles 900 is not so much. In comparison with the conventional device, the misjudgment of the device 1 for judging state of motion of a user of the present invention is minimized so that the waste of social resource and medical resource can be reduced.

In a modified embodiment, in order to provide more bases for the judgment of the motional state of the user, the first sensor 101 of the first sensation module 100 of the device 1 for judging state of motion of a user further includes a first electronic compass (not shown) for generating first directional information, a first air pressure sensation unit (not shown) for generating first air pressure information, a first positioning unit (not shown) for generating first positioning information (such as global satellite positioning system receiving unit) or a first pickup unit (not shown) for generating first audio information (such as microphone) or any combination of the above sensation components. For example, the second processor 103 of the first sensation module 100 receives and combines the first acceleration information, the first angular velocity information and the sensation information generated by any (or several) of the above sensation components to generate the first motional state information.

The second sensor 201 of the second sensation module 200 further includes a second electronic compass (not shown) for generating second directional information, a second air pressure sensation unit (not shown) for generating second air pressure information, a second positioning unit (not shown) for generating second positioning information (such as global satellite positioning system receiving unit) or a second pickup unit (not shown) for generating second audio information (such as microphone) or any combination of the above sensation components. For example, the first processor 203 of the second sensation module 200 receives and combines the second acceleration information, the second angular velocity information and the sensation information generated by any (or several) of the above sensation components to generate the second motional state information. Accordingly, the first processor 203 can compare the first and second motional state information to more precisely judge whether an accident happens to the user.

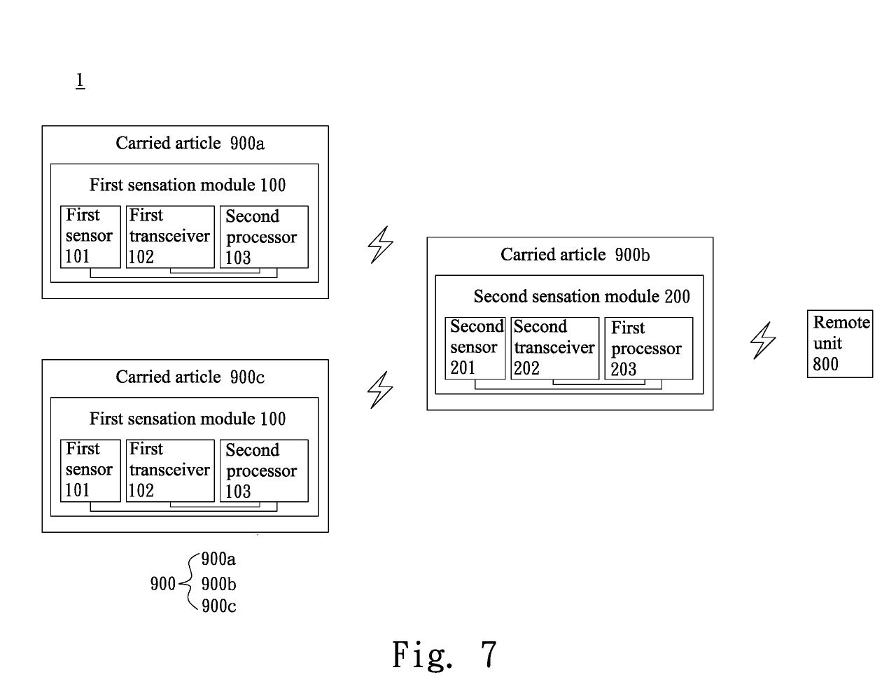

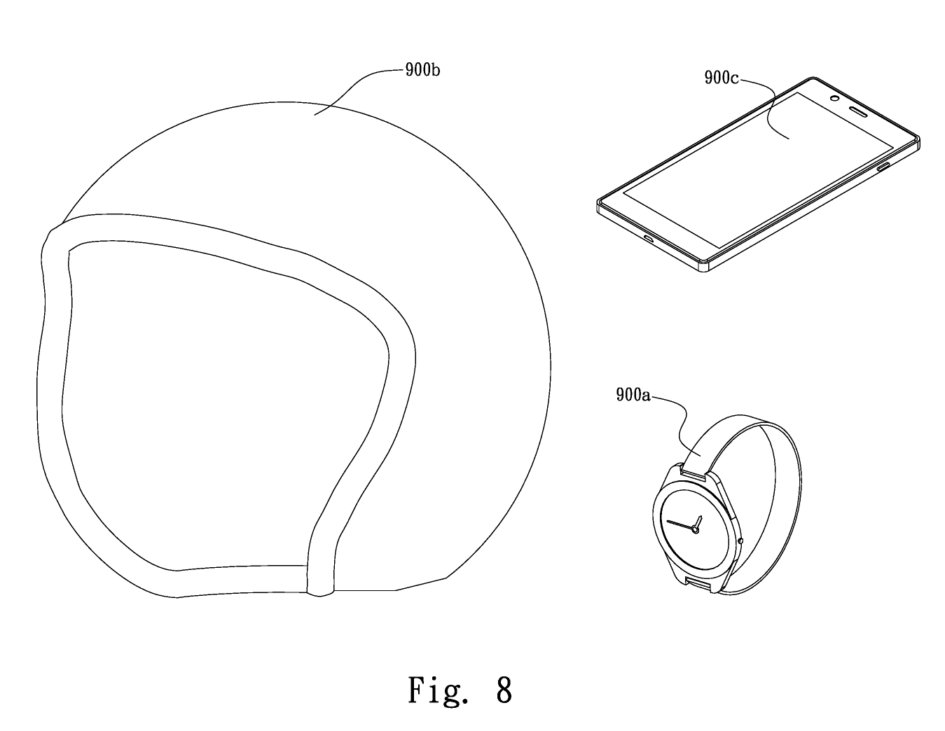

Please now refer to FIGS. 7 and 8. FIG. 7 is a block diagram of a second embodiment of the present invention. FIG. 8 is a perspective view of the second embodiment of the present invention. The second embodiment is substantially identical to the first embodiment in structure, connection relationship and effect. The second embodiment is different from the first embodiment in that there are three carried articles, that is, a watch, a helmet and a mobile device. In addition, the device 1 for judging state of motion of a user has multiple first sensation modules 100. In this embodiment, there are two first sensation modules 100, one of which is disposed on the watch (the carried article 900a), while the other of which is disposed on the mobile device (the carried article 900c). The second sensation module 200 is disposed on the helmet (the carried article 900b). Therefore, in order to more precisely detect the state of motion of a user, the first sensation modules 100 can be disposed on multiple carried articles 900 carried by the user so as to minimize the possibility of misjudgment.

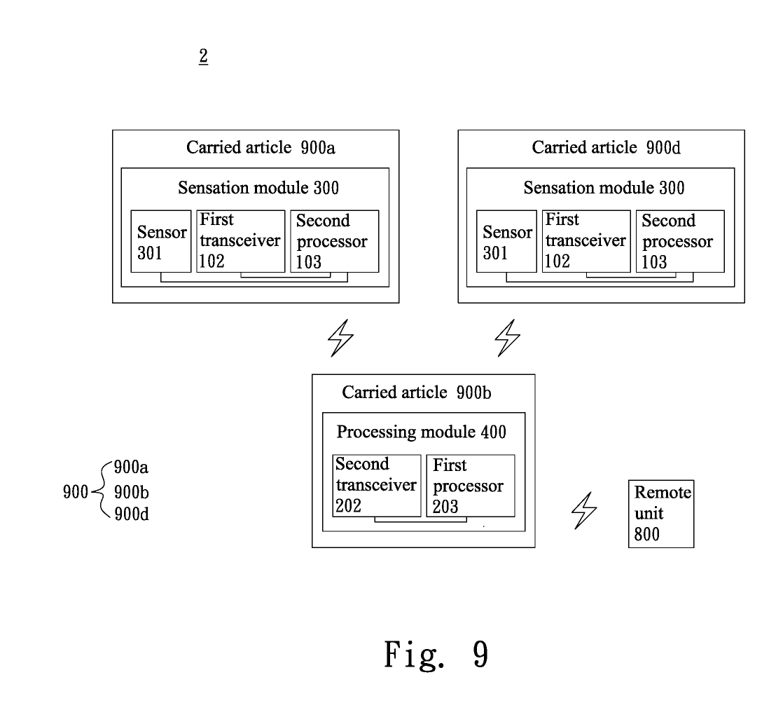

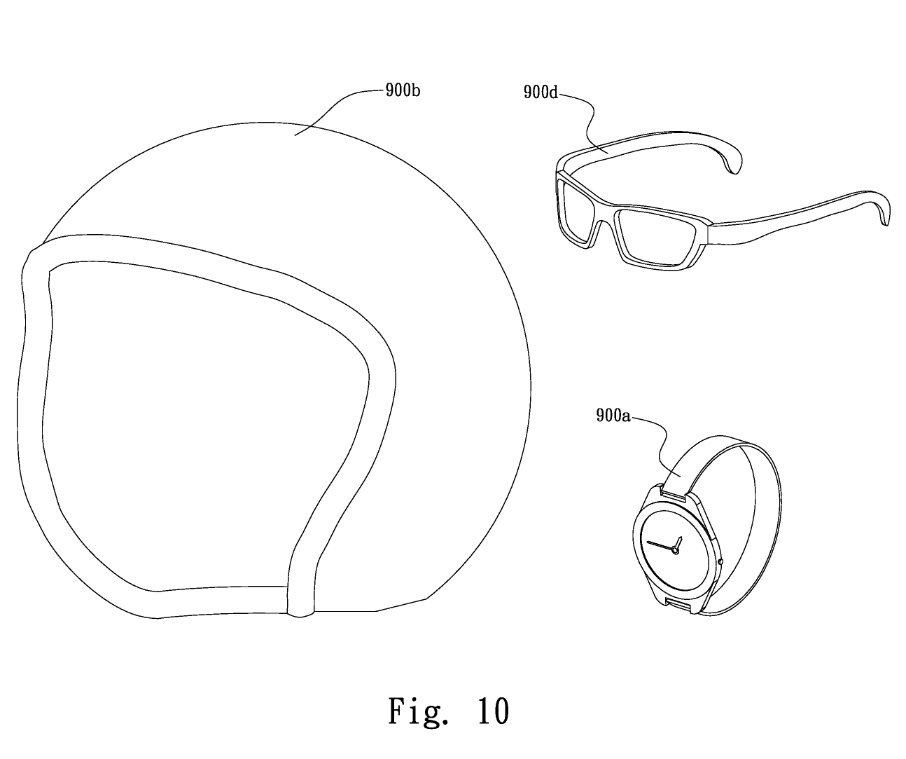

Please now refer to FIGS. 9 and 10 and supplementally to FIG. 3. FIG. 9 is a block diagram of a third embodiment of the present invention. FIG. 10 is a perspective view of the third embodiment of the present invention. In the third embodiment, the device 2 for judging state of motion of a user is disposed on multiple carried articles 900 carried by a user. In this embodiment, there are three carried articles 900, that is, a watch, a helmet and a pair of glasses. In addition, the device 2 for judging state of motion of a user includes multiple sensation modules 300 and a processing module 400. The processing module 400 is disposed on at least one of the carried articles 900 carried by the user. In this embodiment, the processing module 400 is disposed on one carried article 900b (the helmet). In this embodiment, there are, but not limited to, two sensation modules 300 for illustration purposes. In practice, multiple sensation modules 300 can be disposed on multiple carried articles 900 carried by the user. In addition, the carried articles 900 of this embodiment are not limited to the watch, the helmet and the glasses. In practice, the carried articles 900 can be alternatively a mobile device (such as an intelligent mobile phone or tablet) or a wearing accessory (such as a bracelet, anklet or intelligent bracelet) or a wear (such as a work hat or necklace) or any combination of the above mobile device, wearing accessory and wear.

One of the sensation modules 300 is disposed on the watch (the carried article 900a), while the other sensation module 300 is disposed on the glasses (the carried article 900d). In this embodiment, the sensation modules and the processing module are respectively disposed on different carried articles. Only one of the sensation modules or the processing module will be disposed on one carried article. That is, the sensation module and the processing module will not be disposed on the same carried article. In a modified embodiment, the sensation module and the processing module can be disposed on the same carried article. That is, the processing module and one of the sensation modules are disposed on the same carried article 900b (the helmet) and the rest of the sensation modules are respectively disposed on the carried articles 900a, 900d (the watch and the glasses).

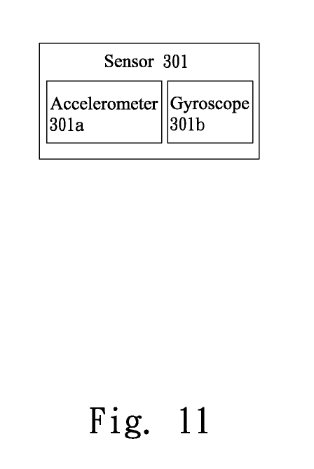

Moreover, each sensation module 300 has a sensor 301, a first transceiver 102 and a second processor 103. The sensor 301 serves to detect the state of motion of the carried articles 900a, 900d. Each sensor 301 includes an accelerometer 301a and a gyroscope 301b (as shown in FIG. 11). The accelerometer 301a of the sensor 301 of one of the sensation modules 300 detects the acceleration of the carried article 900a to generate acceleration information. The gyroscope 301b detects the angular velocity of the carried article 900a to generate angular velocity information. The accelerometer 301a of the sensor 301 of the other of the sensation modules 300 detects the acceleration of the carried article 900d to generate other acceleration information. The gyroscope 301b detects the angular velocity of the carried article 900d to generate other angular velocity information. The second processor 103 is connected to the corresponding sensor 301 and the first transceiver 102. The second processor 103 receives and combines the acceleration information and the angular velocity information to generate motional state information. In this embodiment, the second processor 103 is a microprocessor. In practice, the second processor 103 can be any operation component with operation function, such as a central processing unit (CPU) or a digital signal processor (DSP). The sensation module 300 transmits the motional state information via the first transceiver 102. In this embodiment, the first transceiver 102 is, but not limited to, a Wi-Fi transceiver. In practice, the first transceiver 102 can be any transceiver component with information transmitting/receiving function, such as a Bluetooth, RFID, 3G or 4G transceiver.

The processing module 400 has a first processor 203 and a second transceiver 202. The first processor 203 is connected to the second transceiver 202. The second transceiver 202 receives the motional state information transmitted from the first transceiver 102. The first processor 203 receives and compares the motional state information. If it is found that both the motional state information have instantaneously changed data, the first processor 203 generates notice information. In this embodiment, the first processor 203 is a central processing unit (CPU). In practice, the first processor 203 can be any operation component with operation function, such as a microprocessor or a digital signal processor (DSP). In this embodiment, the second transceiver 202 is, but not limited to, a Wi-Fi transceiver. The second transceiver 202 is coupled with the first transceiver 102. In practice, the second transceiver 202 can be any transceiver component with information transmitting/receiving function, such as a Bluetooth, RFID, 3G or 4G transceiver.

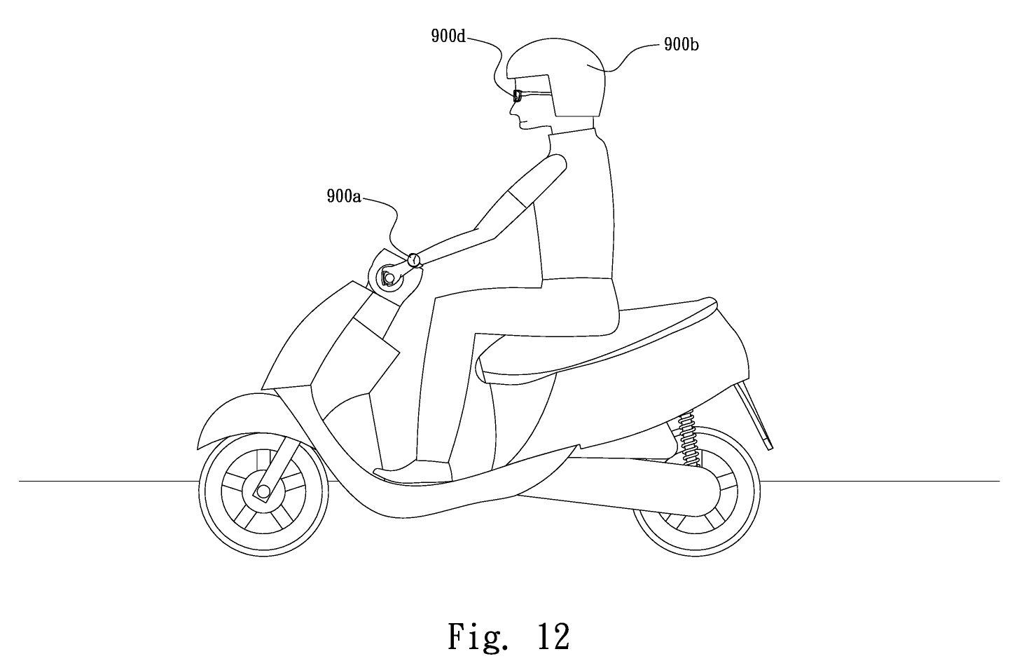

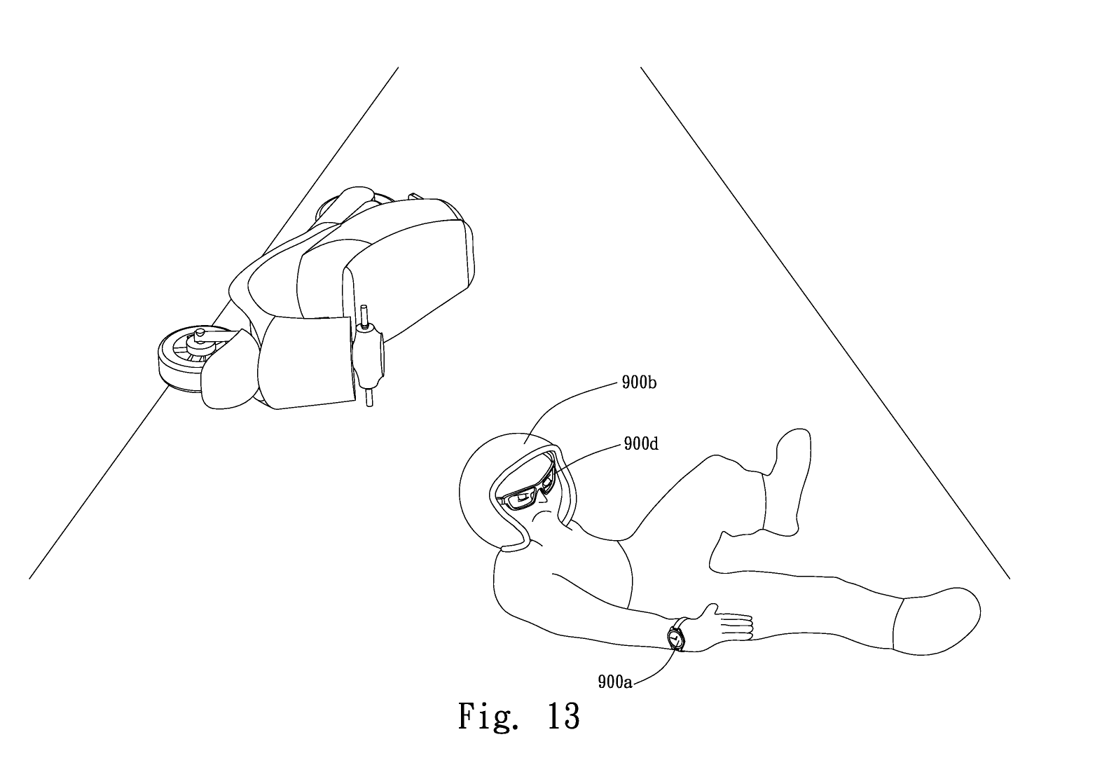

The instantaneously changed data means at a certain point of time (such as at the point of an accident), the sensors 301 detect that the motional states of the carried articles 900a, 900d are instantaneously changed to generate instantaneously changed data. In normal state (without any accident), no instantaneously changed data will be generated. Under such circumstance, the sensors 301 detect that the motional states of the carried articles 900a, 900d are not instantaneously changed. For example, as shown in FIG. 12, in normal state, a user wears a helmet (at least one carried article 900b) and a watch (the carried article 900a) and a pair of glasses (the other carried article 900d) and rides a motorcycle. During the riding, the accelerometer 301a and the gyroscope 301b of the sensor 301 detect that the acceleration and angular velocity of the watch and the glasses periodically up and down and left and right change within a small range. Also, each sensor 301 detects that the periodical changes of the acceleration and angular velocity of the watch and the glasses are substantially identical (no instantaneously changed data are generated). Therefore, the motional state information is substantially identical. As shown in FIG. 13, in case an accident happens to the user during the riding (at a certain point of time), for example, the user is collided or falls down, the periodical changes of the acceleration and angular velocity of the watch and the glasses detected by the sensors 301 will simultaneously greatly change (instantaneously changed data are generated). At this time, the first processor 203 of the processing module 400 compares and finds that both the motional state information simultaneously greatly change (the instantaneously changed data of each motional state information is not certainly identical to each other). Accordingly, it is judged that an accident happens to the user. Under such circumstance, the first processor 203 generates the notice information and transmits the notice information to a remote unit 800 via the second transceiver 202.

In this embodiment, the remote unit 800 is a server end and includes the mobile device or database of another user that regards the server end as an information service providing end and receives the information service from the server end. For example, in case an accident happens to the user, the device 2 for judging state of motion of a user can immediately transmit the notice information to the remote unit 800. Then, the remote unit 800 transmits the notice information to the mobile phone of the user's family or the computer of a medical station. Accordingly, the device 2 for judging state of motion of a user of the present invention can get the user a quicker medical care after the accident. This can increase the survival rate. In a modified embodiment, the remote unit 800 can be a mobile device (such as an intelligent mobile phone or a tablet) or a device capable of receiving and reading the notice information (such as a computer, a notebook or a tablet). For example, the remote unit 800 can be an intelligent mobile phone of the user's family. In this case, the intelligent mobile phone of the user's family can directly receive the notice information transmitted from the second transceiver 202 on the user to let the user's family at the first moment know that an accident happens to the user and immediately help the user.

In addition, when riding a motorcycle, the user wears the pair of glasses (the carried article 900d) on his/her head and wears the watch (the carried article 900a) on his/her hand. During the riding, one of the parts (such as the head and hand) of the user that wear the carried articles 900 may make a motion such as turn the head or raise the hand. At this time, the first processor 203 of the processing module 400 will compare the motional state information. In case the motional state information generated by one of the glasses and the watch is found changed (it is found that one of the glasses and the watch generates instantaneously changed data) and only the sensor of one carried article 900 detects the motional state of the carried article 900 is changed, the first processor 203 will judge that no accident happens to the user since only one motional state is changed. Alternatively, in case the parts of the user that wear the carried articles 900 make a motion such as turn the head or raise the hand and the change of the motional state information due to the motion is not so much, the first processor 203 will judge that no accident happens to the user since the change of the motional state of the carried articles 900 is not so much. In comparison with the conventional device, the misjudgment of the device 2 for judging state of motion of a user of the present invention is minimized so that the waste of social resource and medical resource can be reduced.

In a modified embodiment, in order to provide more bases for the judgment of the motional state of the user, the sensor 301 of each sensation module 300 of the device 2 for judging state of motion of a user further includes an electronic compass (not shown) for generating directional information, an air pressure sensation unit (not shown) for generating air pressure information, a positioning unit (not shown) for generating positioning information (such as global satellite positioning system receiving unit) or a pickup unit (not shown) for generating audio information (such as microphone) or any combination of the above sensation components. Alternatively, each sensation module 300 can include different combinations of sensation components. For example, one of the sensation modules 300 (such as disposed on the watch) includes an accelerometer, a gyroscope and an electronic compass, while the other of the sensation modules 300 (such as disposed on the glasses) includes an accelerometer, a gyroscope, an air pressure sensation unit and a positioning unit. For example, the second processor 103 of each sensation module 300 receives and combines the acceleration information, the angular velocity information and the sensation information generated by any (or several) of the above sensation components to respectively generate the motional state information. Accordingly, the first processor 203 can compare the motional state information to more precisely judge whether an accident happens to the user.

According to the above arrangement, the device for judging state of motion of a user of the present invention can get the user a quicker medical care after the accident and increase the survival rate. On the other hand, in comparison with the conventional device, the misjudgment of the device for judging state of motion of a user of the present invention is minimized so that the waste of social resource and medical resource can be reduced.

The present invention has been described with the above embodiments thereof and it is understood that many changes and modifications in the above embodiments can be carried out without departing from the scope and the spirit of the invention that is intended to be limited only by the appended claims.

What is claimed is:

1. A device for judging state of motion of a user, the device being disposed on multiple carried articles carried by a user, the device for judging state of motion of a user comprising:
at least one first sensation module disposed on at least one of the carried articles, the first sensation module having a first sensor and a first transceiver, the first sensor serving to detect the state of motion of the at least one carried article of the multiple carried articles to generate first motional state information, the first motional state information being transmitted via the first transceiver; and
a second sensation module disposed on the other of the carried articles, the second sensation module having a second sensor, a second transceiver and a first processor, the first processor being connected to the second sensor and the second transceiver, the second sensor serving to detect the state of motion of the other carried article of the multiple carried articles to generate second motional state information and transmit the second motional state information to the first processor, the first processor receiving the first motional state information of the first sensation module via the second transceiver and comparing the first and second motional state information to find that both the first and second motional state information have instantaneously changed data to generate notice information, the notice information being transmitted to a remote unit via the second transceiver.

2. The device for judging state of motion of a user as claimed in claim 1, wherein the carried articles are a mobile device or a wearing accessory or a wear or any combination thereof.

3. The device for judging state of motion of a user as claimed in claim 1, wherein the first sensation module further has a second processor connected to the first sensor and the first transceiver, the second processor transmitting the first motional state information to the second sensation module via the first transceiver.

4. The device for judging state of motion of a user as claimed in claim 1, wherein the first sensor includes a first accelerometer and a first gyroscope, the first accelerometer detecting the acceleration of the at least one carried article of the multiple carried articles to generate first acceleration information, the first gyroscope detecting the angular velocity of the at least one carried article of the multiple carried articles to generate first angular velocity information, the first acceleration information and the first angular velocity information being combined to generate the first motional state information.

5. The device for judging state of motion of a user as claimed in claim 1, wherein the second sensor includes a second accelerometer and a second gyroscope, the second accelerometer detecting the acceleration of the other carried article of the multiple carried articles to generate second acceleration information, the second gyroscope detecting the angular velocity of the other carried article of the multiple carried articles to generate second angular velocity information, the second acceleration information and the second angular velocity information being combined to generate the second motional state information.

6. The device for judging state of motion of a user as claimed in claim 4, wherein the first sensor further includes a first electronic compass, a first air pressure sensation unit, a first positioning unit or a first pickup unit, the first electronic compass serving to generate first directional information, the first air pressure sensation unit serving to generate first air pressure information, the first positioning unit serving to generate first positioning information, the first pickup unit serving to generate first audio information.

7. The device for judging state of motion of a user as claimed in claim 5, wherein the second sensor further includes a second electronic compass, a second air pressure sensation unit, a second positioning unit or a second pickup unit, the second electronic compass serving to generate second directional information, the second air pressure sensation unit serving to generate second air pressure information, the second positioning unit serving to generate second positioning information, the second pickup unit serving to generate second audio information.

8. The device for judging state of motion of a user as claimed in claim 1, wherein the remote unit is a server end or a mobile device.

9. A device for judging state of motion of a user, the device being disposed on multiple carried articles carried by a user, the device for judging state of motion of a user comprising:
multiple sensation modules disposed on the multiple carried articles, each sensation module having a sensor and a first transceiver, the sensors serving to detect the state of motion of the multiple carried articles to generate multiple motional state information, the motional state information being transmitted via first transceivers of the sensation modules; and
a processing module disposed on at least one of the multiple carried articles, the processing module having a first processor and a second transceiver connected to the first processor, the second transceiver receiving the motional state information transmitted from the first transceiver, the first processor receiving the motional state information and comparing the motional state information to find that both the motional state information have instantaneously changed data to generate notice information, the notice information being transmitted to a remote unit via the second transceiver.

10. The device for judging state of motion of a user as claimed in claim 9, wherein the carried articles are a mobile device or a wearing accessory or a combination thereof.

11. The device for judging state of motion of a user as claimed in claim 9, wherein each sensation module has a second processor connected to the sensor and the first transceiver, the second processor transmitting the motional state information to the processing module via the first transceiver.

12. The device for judging state of motion of a user as claimed in claim 9, wherein the sensor includes an accelerometer and a gyroscope, the accelerometer detecting the acceleration of the carried article to generate acceleration information, the gyroscope detecting the angular velocity of the carried article to generate angular velocity information, the acceleration information and the angular velocity information being combined to obtain the motional state information.

13. The device for judging state of motion of a user as claimed in claim 12, wherein the sensor further includes an electronic compass, an air pressure sensation unit, a positioning unit or a pickup unit, the electronic compass serving to generate directional information, the air pressure sensation unit serving to generate air pressure information, the positioning unit serving to generate positioning information, the pickup unit serving to generate audio information.

14. The device for judging state of motion of a user as claimed in claim 9, wherein the remote unit is a server end or a mobile device.
